(19) United States
(12) Patent Application Publication
(10) Pub. No.: US 2005/0249757 A1
(43) Pub. Date: Nov. 10, 2005

(54) Pharmaceutical cream formulations

(75) Inventors: Muthaiyyan Esakki Kannan, Kopar Khairane (IN); Suma Prakasan Nair, Dombivli (IN); Anandi Krishnan, Vashi (IN)

Correspondence Address: DILWORTH & BARRESE, LLP, 333 EARLE OVINGTON BLVD., UNIONDALE, NY, 11553, US

(73) Assignee: Glenmark Pharmaceuticals Limited, Mumbai (IN)

(21) Appl. No.: 11/124,334

(22) Filed: May 6, 2005

Related U.S. Application Data
(60) Provisional application No. 60/568,813, filed on May 6, 2004.

(51) Int. Cl. A61K 31/4745; A61K 9/00

(52) U.S. Cl. 424/400; 514/183; 514/291

(57) ABSTRACT

A pharmaceutical cream composition is provided comprising (a) a therapeutically effective amount of one or more active pharmaceutical ingredients comprising one or more macrolide related immunosuppressants or pharmaceutically acceptable salts or esters thereof; (b) one or more cream forming agents; and (c) an effective amount of one or more skin penetration enhancers capable of percutaneous delivery of the macrolide related immunosuppressant through the skin. Also provided is a process for its preparation and methods for delivering a macrolide related immunosuppressant through the skin of a mammal in order to treat conditions situated on and beneath the skin.

### CROSS-REFERENCE TO RELATED APPLICATIONS

This application claims the benefit under 35 U.S.C. §119 to Provisional Application No. 60/568,813, filed May 6, 2004 and entitled “TOPICAL MACROLIDE PHARMACEUTICAL CREAM FORMULATIONS”, the contents of which are incorporated by reference herein.

### BACKGROUND OF THE INVENTION

1. Technical Field

The present invention relates generally to pharmaceutical cream formulations.

2. Description of the Related Art

The administration of drugs and other biological materials to the bloodstream via a transdermal route or to the localized site of action has received much attention in recent years. The skin of an average adult covers generally more than two square meters of surface area and receives about one third of all blood circulating through the body. It is elastic, rugged, and generally self generating. The skin consists of three layers: the stratum corneum, the epidermis, and the dermis.

The stratum corneum represents the rate-limiting step in diffusion of chemicals through the skin. The stratum corneum is composed of dead, keratinized, metabolically inactive cells, which are closely packed together, and consists of an amorphous matrix of mainly lipoid and non fibrous protein within which keratin filaments are distributed. The cells of the stratum corneum generally contain about 20% water, while the cells below, in the stratum germinivatum contain about 70% water. The stratum corneum does not become hydrated readily. Thus, transdermal permeation is primarily controlled by diffusion through the stratum corneum.

Due to availability of large surface area, easy accessibility, application dynamics and the noninvasive nature of the therapy, topical administration of drugs has long been considered a promising route of drug delivery whether the bioavailability desired is systemic, dermal, regional or localized. The topical mode of drug delivery provides many advantages over customarily used routes of administration. First, it bypasses the portal circulation and thereby the hepatic first pass metabolism. Second, topical delivery avoids the problems of variable systemic absorption and metabolism. Third, it potentially reduces gastrointestinal irritation associated with oral administration. Further, it avoids the risks and patient noncompliance associated with parenteral treatment.

The topical delivery route offers continuity of drug administration, permits use of therapeutic agents with short biological half lives, provides treatment of cutaneous manifestations of diseases usually treated systemically delivers medication directly into the systemic circulation and fosters ease of use and total patient compliance.

Pharmaceutical creams are semisolid preparations containing one or more active pharmaceutical agents (API) dissolved or dispersed in either an oil-in-water emulsion or in another type of water-washable base. Oil-in-water emulsions containing a large percentage of water and stearic acid are sometimes referred to as “vanishing creams.” After application of the cream, the water evaporates leaving behind a thin residue film of stearic acid.

Several pharmaceutical compositions are described in literature for topical application of macrolide related immunosuppressant drugs which are known to exhibit efficacy in the treatment of autoimmune related skin conditions.

One example is Protopic® (tacrolimus) ointment which contains tacrolimus, a macrolide immunosuppressant produced by Streptomyces tsukubaensis. It is typically utilized for topical dermatologic use only. Chemically, tacrolimus is designated as [3S-[3R*[E(1S*,3S*,4S*)],4S*,5R*,8S*,9E,12R*,14R*,15S*,16R*,18S*,19S*,26aR*]]-5,6,8,11,12,13,14,15,16,17,18,19,24,25,26,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxaazacyclotricosine-1,7,20,21(4H,23H)-tetrone, monohydrate as shown in FIG. I: 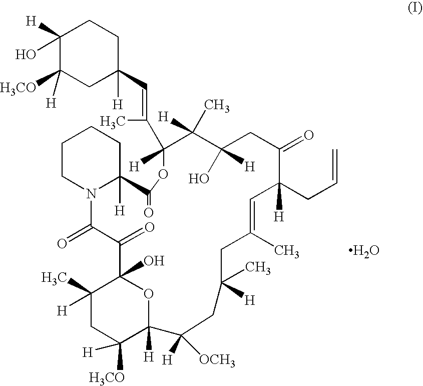
Tacrolimus has an empirical formula of C₄₄H₆₉NO₁₂.H₂O and a formula weight of 822.03. It has been demonstrated that tacrolimus inhibits T lymphocyte activation by first binding to an intracellular protein, FKBP-12. A complex of tacrolimus-FKBP-12, calcium, calmodulin, and calcineurin is then formed and the phosphatase activity of calcineurin is inhibited. This effect has been shown to prevent the dephosphorylation and translocation of nuclear factor of activated T-cells (NF-AT), a nuclear component thought to initiate gene transcription for the formation of lymphokines (such as interleukin-2, gamma interferon). Tacrolimus also inhibits the transcription for genes which encode IL-3, IL-4, IL-5, GM-CSF, and TNF-Ε, all of which are involved in the early stages of T-cell activation. Additionally, tacrolimus has been shown to inhibit the release of pre-formed mediators from skin mast cells and basophils, and to down regulate the expression of FceRI on Langerhans cells.

Tacrolimus is indicated for short-term and intermittent long-term therapy in the treatment of patients with moderate to severe atopic dermatitis in whom the use of alternative, conventional therapies are deemed inadvisable because of potential risks, or in the treatment of patients who are not adequately responsive to or are intolerant of alternative, conventional therapies. See, e.g., Physician's Desk Reference, “Protopic,” 58th Edition, p. 1327-1330 (2003).

Another example is Elidel® (pimecrolimus) Cream 1% which contains the compound pimecrolimus, the 33-epi-chloro-derivative of the macrolactam ascomycin. Chemically, pimecrolimus is (1R,9S,12S,13R,14S,17R,18E,21 S,23S,24R,25S,27R)-12-[(1E)-2-{(1R,3R,4S)-4-chloro-3-methoxycyclohexyl}-1-methylvinyl]-17-ethyl-1,14-dihydroxy-23,25-dimethoxy-13,19,21,27-tetramethyl-11,28-dioxa-4-aza-tricyclo[22.3.1.04,9]octacos-18-ene-2,3,10,16-tetraone as shown in FIG. II: 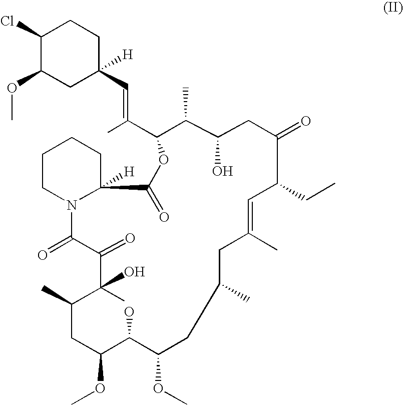
Pimecrolimus has the empirical formula C₄₃H₆₈ClNO₁₁ and a molecular weight of 810.47. Pimecrolimus has been demonstrated to bind with high affinity to macrophilin-12 (FKBP-12) and inhibit the calcium-dependent phosphatase, calcineurin. As a consequence, it inhibits T-cell activation by blocking the transcription of early cytokines. In particular, pimecrolimus inhibits at nanomolar concentrations Interleukin-2 and interferon gamma (Th1-type) and Interleukin-4 and Interleukin-10 (Th2-type) cytokine synthesis in human T cells. In addition, pimecrolimus prevents the release of inflammatory cytokines and mediators from mast cells in vitro after stimulation by antigen/IgE. Pimecrolimus is indicated for short-term and intermittent long-term therapy in the treatment of mild to moderate atopic dermatitis. See, e.g., Physician's Desk Reference, “Elidel,” 58th Edition, p. 2250-2252 (2003).

Yet another example is Rapamune® (sirolimus) which is an immunosuppressive agent. Sirolimus is a macrocyclic lactone produced by Streptomyces hygroscopicus. The chemical name of sirolimus (also known as rapamycin) is (3S,6R,7E,9R,10R,12R,14S,15E,17E,19E,21S,23S,26R,27R,4aS)-9,10,12,13,14,21,22,23,24,25,26,27,32,33,34,34a-hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]oxaazacyclohentriacontine-1,5,11,28,29(4H,6H,31H)-pentone as shown in FIG. III: 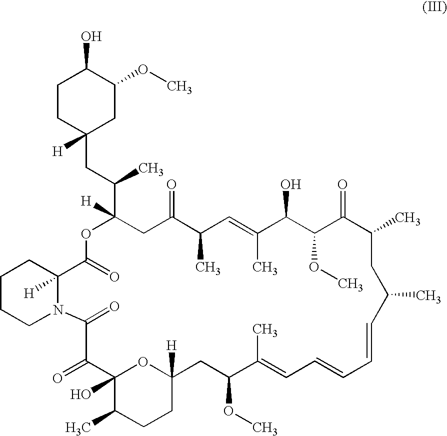
Sirolimus has a molecular formula of C₅₁H₇₉NO₁₃ and its molecular weight is 914.2. Sirolimus inhibits T lymphocyte activation and proliferation that occurs in response to antigenic and cytokine (Interleukin [IL]-2, IL-4, and IL-15) stimulation by a mechanism that is distinct from that of other immunosuppressants. Sirolimus also inhibits antibody production. In cells, sirolimus binds to the immunophilin, FK Binding Protein-12 (FKBP-12), to generate an immunosuppressive complex. The sirolimus:FKBP-12 complex has no effect on calcineurin activity. This complex binds to and inhibits the activation of the mammalian Target Of Rapamycin (mTOR), a key regulatory kinase. This inhibition suppresses cytokine-driven T-cell proliferation, inhibiting the progression from the G1 to the S phase of the cell cycle. Sirolimus is indicated for the prophylaxis of organ rejection in patients receiving renal transplants. See, e.g., Physician's Desk Reference, “Rapamune,” 58th Edition, p. 3483-3490 (2003).

### SUMMARY OF THE INVENTION

In accordance with one embodiment of the present invention, a pharmaceutical cream composition is provided comprising (a) a therapeutically effective amount of one or more active pharmaceutical ingredients comprising one or more macrolide related immunosuppressants or pharmaceutically acceptable salts or esters thereof; (b) one or more cream forming agents; and (c) an effective amount of one or more skin penetration enhancers capable of percutaneous delivery of the macrolide related immunosuppressant through the skin.

In accordance with a second embodiment of the present invention, a process for preparing a pharmaceutical cream composition is provided comprising (a) dissolving a therapeutically effective amount of one or more active pharmaceutical ingredients comprising one or more macrolide related immunosuppressants or pharmaceutically acceptable salts or esters thereof in one or more penetration enhancers to form a solution; (b) providing a cream forming phase comprising one or more cream forming agents; and (c) mixing the product of step (a) with the product of step (b).

In accordance with a third embodiment of the present invention, a method for delivering one or more macrolide related immunosuppressants or pharmaceutically acceptable salts or esters thereof through the skin in order to treat conditions situated on and beneath the skin is provided, the method comprising the step of topically administering an effective amount of a pharmaceutical cream composition comprising (a) a therapeutically effective amount of one or more active pharmaceutical ingredients comprising one or more macrolide related immunosuppressants or pharmaceutically acceptable salts or esters thereof; (b) a cream forming agent; and (c) an effective amount of one or more skin penetration enhancers capable of percutaneous delivery of the macrolide related immunosuppressant through the skin.

### DEFINITIONS

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

By “pharmaceutically acceptable” is meant those salts and esters which are, within the scope of sound medical judgment, suitable for use in contact with the tissues of humans and lower animals without undue toxicity, irritation, allergic response and the like, commensurate with a reasonable benefit/risk ratio, and effective for their intended use. Representative acid additions salts include the hydrochloride, hydrobromide, sulphate, bisulphate, acetate, oxalate, valerate, oleate, palmitate, stearate, laurate, borate, benzoate, lactate, phosphate, tosylate, mesylate, citrate, maleate, fumarare, succinate, tartrate, ascorbate, glucoheptonate, lactobionate, lauryl sulphate salts and the like. Representative alkali or alkaline earth metal salts include the sodium, calcium, potassium and magnesium salts, and the like.

The term “subject” or “a patient” or “a host” as used herein refers to mammalian animals, preferably human.

As used herein the term “antioxidant” is intended to mean an agent who inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisulfite and other such materials known to those of ordinary skill in the art.

As used herein, the term “buffering agent” is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

As used herein, the term “binders” is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ f127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

As used herein, the term “wetting agent” is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

One aspect of the present invention provides a therapeutic macrolide related immunosuppressant containing pharmaceutical cream composition containing at least (a) a therapeutically effective amount of one or more active pharmaceutical ingredients comprising one or more macrolide related immunosuppressants or pharmaceutically acceptable salts or esters thereof; (b) a cream forming agent; and (c) an effective amount of one or more skin penetration enhancers capable of percutaneous delivery of the macrolide related immunosuppressant through the skin. The terms “active”, “drug”, and “active pharmaceutical ingredient” are used interchangeable herein. The macrolide related immunosuppressants for use herein can be any known macrolide immunosuppressants and includes, but are not limited to, tacrolimus, pimecrolimus, sirolimus, acsomycin, everolimus, pharmaceutically acceptable salts or esters thereof, derivatives thereof, and the like and mixtures thereof.

Pharmaceutical cream-forming components are well known and may be, for example, viscous liquid or semisolid emulsions, either oil-in-water or water-in-oil emulsions and related formulations comprising two or more phases. Generally, cream bases are water-washable, and contain an oil phase, an aqueous phase and optionally one or more components such as, for example, emollients, emulsifiers, thickeners, preservatives and the like and mixtures thereof.

Suitable emollients include, but are not limited to, long chain alcohols, e.g., cetyl alcohol, stearyl alcohol and cetearyl alcohol; hydrocarbons, e.g., petrolatum and light mineral oil; or acetylated lanolin and the like and mixtures thereof. The total amount of emollient in a cream of the present invention can range from about 5% to about 40% w/w.

The emulsifiers are conventional emulsifiers known in the art and generally are one or more nonionic, anionic, cationic or amphoteric surfactants. Suitable nonionic surfactants include, by way of example, polysorbate 60 (available from ICI Americas), sorbitan monostearate, polyglyceryl-4 oleate, and polyoxyethylene(4)lauryl ether and the like and mixtures thereof. Generally the amount of each emulsifier can range from about 0.01 to about 10% w/w.

Pharmaceutically acceptable thickeners such as Veegum™ K (available from R. T. Vanderbilt Company, Inc.), and long chain alcohols (e.g., cetyl alcohol, stearyl alcohol or cetearyl alcohol) and the like and mixtures thereof can be used. Preservatives such as methylparaben, propylparaben and benzyl alcohol can also be present in a cream of the present invention. The appropriate amount of such preservative(s) is known to those skilled in the art. Optionally, a cream of the present invention can also contain a humectant such as glycerin. It is known to those skilled in the art that a single ingredient can perform more than one function in a cream, e.g., cetyl alcohol can serve both as an emollient and as a thickener.

Generally, a cream consists of an oil phase and a water phase mixed together to form an emulsion. The amount of water present in a cream of the present invention is about 1 to about 80% w/w and preferably about 35 to about 65% w/w. The oil phase, also sometimes called the “internal” phase, is generally formed from, for example, petrolatum and a fatty alcohol containing from 6 to about 44 carbon atoms, e.g., cetyl alcohol, stearyl alcohol etc. and mixtures thereof. The aqueous phase usually, although not necessarily, exceeds the oil phase in volume, and generally contains a humectant.

“Penetration enhancement” or “permeation enhancement” as used herein relates to an increase in the permeability of the skin or mucosal tissue to the selected pharmacologically active agent, which in turn gives rise to an increase in the rate at which the drug permeates into and/or through the skin or mucosal tissue. Accordingly, the skin penetration enhancers herein are employed to improve the permeability of an active pharmaceutical ingredient through the skin. Exemplary penetration enhancers include, by way of example and without limitation, volatile organic solvents (e.g. alcohols such as ethanol), nonvolatile organic solvents (e.g. amides such as pyrrolidones; polyol ethers such as glycol ethers; polyols such as glycols; and derivatives thereof) and the like and mixtures thereof.

Suitable volatile organic solvents include, but are not limited to, aliphatic, cycloaliphatic and/or aromatic-aliphatic alcohols, each of which is monohydric or polyhydric, alcohol/water mixtures, saturated and/or unsaturated fatty alcohols which each contain from about 8 to about 18 carbon atoms, saturated and/or unsaturated fatty acids which each from about 8 to about 18 carbon atoms and/or esters thereof and the like and mixtures thereof. Useful alcohols are those having from 1 to about 20 carbon atoms, e.g., ethanol, isopropyl alcohol, etc. Topical ethanol solutions can also be used as a penetration enhancer at a concentration ranging from about 1% v/v to about 90% v/v. See, e.g., A. Kibbe, Handbook of Pharmaceutical Excipients, 3rd Ed. (2000). The amounts of ethanol may vary greatly, even outside the ranges given by the Handbook of Pharmaceutical Excipients. In the present invention, an alcohol can be used as co-solvent cum penetration enhancer for the API. While not being bound to any particular theory, there are two theories supporting the use of alcohol as skin penetration enhancer. First, the alcohol evaporates fast and concentrates the drug in the residual formulation that remains on the skin. It is believed that some thermodynamic activity will drive the drug into the stratum corneum. Second, the alcohol alters the physical integrity of the stratum corneum barrier resulting in an increase in the ability of the drug to penetrate the skin. Commercially, denatured alcohol such as SDA 40 is often used in place of Alcohol USP (ethanol), and it may be used here also. A preferred alcohol is ethanol.

Suitable amides for use as nonvolatile organic solvents include, but are not limited to, N,N-dimethyl acetamide (DMA) N,N-diethyl toluamide, N,N-dimethyl formamide, N,N-dimethyl octamide, N,N-dimethyl decamide, and the like; pyrrolidone derivatives such as N-alkylpyrrolidones (e.g., N-methyl-2-pyrrolidones), vinyl pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones, and the like and mixtures thereof. The present invention preferably employs N-methyl-2-pyrrolidone as a nonvolatile type of penetration enhancer. N-methyl-2-pyrrolidone is commercially available as Pharmasolve® (available from ISP Corp. of Wayne, N.J.), a safe and compatible material. Generally, Pharmasolve® increases the water solubility of insoluble drugs and can therefore develop insoluble drugs into topical products.

Solubility enhancement can be attributed to three parameters: nonpolar molecular dispersion, polar type chemical bonding and hydrogen bonding. N-methyl-2-pyrrolidone can undergo a large number of chemical reactions at various positions on the pyrrolidone ring. The enhanced solubility can be attributed to a complexing action with the nitrogen and carbonyl reactive centers of the molecule. A preferred N-methyl-2-pyrrolidone is Pharmasolve® which is a drug solubilizer and therefore enhances the bioavailability of topical formulations. It can increase the solubility of many drugs that are not water-soluble thereby enhancing their physico-chemical stability and bioavailability.

Pharmasolve's® favorable safety profile makes it a solubilizer of choice for use in pharmaceutical formulations. Furthermore, in contrast to other drug solubilizing agents, Pharmasolve® has a favorable toxicity profile, making it a suitable candidate for use in a variety of topical dosage forms. Pharmasolve® is practically nonirritating to rabbit skin. (PII=0.5). According to the manufacturer's brochure, when Pharmasolve® was analyzed in a repeated insult patch test using 50 human subjects and a total of 15 applications, N-methyl-2-pyrrolidone was neither a primary dermal irritant nor a sensitizer.

Polyol ethers for use herein can be C₂-C₃₀ polyol ethers containing from 2 to about 10 hydroxyl groups. Representative of the polyol ethers are glycol ethers which include, by way of example and without limitation, ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether, polyethylene glycol, polyethylene glycol dodecyl ether, diethylene glycol monoethyl ether, polyethylene glycol-8-glyceryl caprylate and the like and mixtures thereof.

Diethylene glycol monoethyl ether, commercially available as Transcutol® (available from Gattefosse of St Priest Mi-Plaine, France). Transcutol® can solubilize hydrophobic materials. The increased drug flux across the stratum corneum is explained by the diffusion of Transcutol® into it, thereby changing the ability of the drug to penetrate the intercellular space. It is believed that the primary role of Transcutol® is the modification of the thermodynamic activity of the drug. By direct action on its solubility, Transcutol® favors the passage of larger quantities of the drug into the stratum corneum allowing a greater solubilization in the aqueous domains of the tissue. Currently, there are various clinical studies conducted in the U.S. using Transcutol® in topical products (Phase I, II and III clinical trials).

Polyols for use herein can be C₂-C₃₀ polyols containing from 2 to about 10 hydroxyl groups. Suitable polyols according to the present invention include, but are not limited to, ethylene glycol, propylene glycol, butylene glycol, hexylene glycol, propylene glycol monocaprylate and mixtures thereof. Propylene glycol is widely used as a solvent or a co-solvent in topicals ranging from about 1% to about 90%. It is also used as a humectant in an amount of about 15%. See, e.g., A. Kibbe, Handbook of Pharmaceutical Excipients, 3rd Ed. (2000). In hydro alcoholic gel formulations, alcohol evaporates, propylene glycol and water forms a saturated solution of drug, which enhances flux of drug across the skin.

In one embodiment, the skin penetration enhancers for use in the compositions of the present invention will be the amides such as the foregoing pyrrolidones and will include one or more solvents that are pharmaceutically acceptable for application to skin or exposed tissue of a non-human. Suitable solvents include, but are not limited to, C₁-C₄ alcohols, C₁-C₄ alkylene glycols, C₁-C₄ polyalcohols, C₁-C₄ polyalkylene glycols, sorbates, polysorbates, benzyl alcohol, triglycerides, and water. Specific examples of suitable components for the solvent mixture include propylene glycol, glycerin, ethanol, isopropyl alcohol and the like. Specifically, propylene glycol, glycerin, isopropyl alcohol, ethanol, and the like, are recognized in the art as safe for topical application to non-human skin and/or exposed tissue.

For example, propylene glycol can serve as a moisturizer and can produce a pleasant emollient feel when applied to the skin. Furthermore, propylene glycol also has the added advantage of being a mild germicide. However, in excessive concentrations the germicidal properties can potentially irritate sensitive skin.

In one example of a suitable embodiment, the solvent mixture includes an alcohol such as ethanol in an amount of about 1% to about 90% w/w and preferably from about 5% to about 50% w/w and a glycol such as propylene glycol in an amount of about 1% to about 90% w/w and preferably from about 5% to about 50% w/w.

The active pharmaceutical ingredient(s) will be present in an amount effective to prevent, treat or aid in the healing of a human skin or tissue disorder. The precise amount of the active pharmaceutical ingredient is dependent upon both the disorder and the human being treated and optimization would therefore involve only routine experimentation. Generally, the amount of the active pharmaceutical ingredient present in the topical pharmaceutical compositions of the present invention can range from about 0.01% to about 5% w/w and preferably from about 0.02% to about 2% w/w. The penetration enhancer will ordinarily be present in an amount sufficient to enhance the penetration of the macrolide related immunosuppressant into the skin. The specific amount varies necessarily according to the desired release rate and specific form of the macrolide related immunosuppressant used. Generally, this amount can range from about 0.01% to about 30% w/w and preferably from about 1% to about 10% w/w.

If desired, the topical pharmaceutical compositions of the present invention can contain additional active pharmaceutical ingredients other than the aforementioned macrolide related immunosuppressants depending on the particular condition being treated. The topical pharmaceutical compositions of the present invention can also include one or more pharmaceutically acceptable excipients that are typically used in the art for locally applied semisolid cream dosage forms. Suitable pharmaceutically acceptable excipients include, but are not limited to, antioxidants, buffering agents, binders, wetting agents, microbial preservatives, stabilizers and the like and mixtures thereof.

The topical pharmaceutical composition herein can be formulated into any suitable formulation such as, for example, immediate release formulation, controlled release formulation, fast melt formulation, delayed release formulation, extended release formulation, mixed release formulations such as immediate release and controlled release formulations and the like.

The pharmaceutical cream compositions of the present invention can be obtained by (a) dissolving a therapeutically effective amount of one or more active pharmaceutical ingredients containing one or more macrolide related immunosuppressants or pharmaceutically acceptable salts or esters thereof in one or more penetration enhancers to form a first solution; (b) providing a cream forming phase containing one or more cream forming agents; and (c) mixing the product of step (a) with the product of step (b).

Another embodiment of the present invention is a method for delivering a macrolide related immunosuppressant through the skin of a mammal in order to treat conditions situated on and beneath the skin including at least the step of topically administering to the skin of the mammal an effective amount of a pharmaceutical cream composition comprising (a) a therapeutically effective amount of one or more active pharmaceutical ingredients comprising one or more macrolide related immunosuppressants or pharmaceutically acceptable salts or esters thereof; (b) one or more cream forming agents; and (c) an effective amount of one or more skin penetration enhancers capable of percutaneous delivery of the macrolide related immunosuppressant through the skin. Conditions which can be treated employing the topical pharmaceutical compositions of the present invention include, but are not limited to, treatment of autoimmune related skin conditions, and the like and combinations thereof.

The following examples are provided to enable one skilled in the art to practice the invention and are merely illustrative of the invention. The examples should not be read as limiting the scope of the invention as defined in the claims.

### EXAMPLE 1

### Cream—Formula Composition I

The ingredients used in this example are set forth below in Table 1.

TABLE 1

| | | | |
|---|---|---|---|
| | | | Range which |
| | | % w/w | can be used |
| Ingredients | Category | (in Example 1) | (% w/w) |

Oil Phase

| | | | |
|---|---|---|---|
| Stearic Acid | Oil Base | 7.00 | 5.00-40.00 |
| Stearyl Alcohol | | 5.00 | |
| Cetyl Alcohol | | 2.00 | |
| Methyl Paraben | Preservative | 0.10 | 0.01-2.00 |
| Propyl Paraben | Preservative | 0.05 | 0.01-2.00 |

Aqueous Phase

| | | | |
|---|---|---|---|
| Tacrolimus | API | 0.10 | 0.01-5.00 |
| (as Tacrolimus | | | |
| Monohydrate) | | | |
| N-Methyl Pyr- | Solubilizer & | 5.00 | 0.01-30.00 |
| rolidone | Penetration | | |
| (Pharmasolve) | Enhancer | | |
| Propylene Glycol | Co-solvent | 20.00 | 1.00-80.00 |
| | for the solu- | | |
| | bilizer | | |
| Glycerin | Aqueous Base | 10.00 | 1.00-50.00 |
| Sodium Lauryl | Anionic Emul- | 1.00 | 0.01-10.00 |
| Sulfate | sifier | | |
| Purified Water | Aqueous Base | 49.75 | 1.00-80.00 |
| Total | | 100.00 | |

The composition of this example was prepared as follows:
1. Tacrolimus was dissolved in N-Methyl-Pyrrolidone and one by fourth quantity of Propylene Glycol was added to the solution and kept aside.
2. All of the ingredients in the oil phase were heated together to 70° C. to 75° C.
3. Sodium Lauryl Sulfate was dissolved in Purified Water.
4. The remaining quantities of Propylene Glycol and Glycerin were added to the solution of step no. 3.
5. The solution of step no. 4 was heated to 70° C. to 75° C.
6. The hot solution from step no. 2 was added to the hot solution of step no. 5, slowly under continuous high speed stirring, to obtain a uniform cream base.
7. The cream base was allowed to cool to 40° C.
8. The tacrolimus solution of step no. 1 was added to the cream base of step no. 7 under gentle stirring.
9. After the addition was completed, the mixture was gently stirred for an additional 5 minutes to obtain a pharmaceutical cream composition having optimum and uniform consistency.

### EXAMPLE 2

### Cream—Formula Composition II

The ingredients used in this example are set forth below in Table 2.

TABLE 2

| | | | |
|---|---|---|---|
| | | | Range which |
| | | % w/w | can be used |
| Ingredients | Category | (in Example 2) | (% w/w) |

Oil Phase

| | | | |
|---|---|---|---|
| Stearic Acid | Oil Base | 14.00 | 5.00-40.00 |
| Isopropyl | | 1.00 | |
| Palmitate | | | |
| Cetyl Alcohol | | 1.00 | |
| Methyl Paraben | Preservative | 0.10 | 0.01-2.00 |
| Propyl Paraben | Preservative | 0.05 | 0.01-2.00 |
| Sorbitan | Non Ionic | 2.00 | 0.01-10.00 |
| Monostearate | Emulsifier | | |

Aqueous Phase

| | | | |
|---|---|---|---|
| Tacrolimus | API | 0.10 | 0.01-5.00 |
| (as Tacrolimus | | | |
| Monohydrate) | | | |
| N-Methyl Pyr- | Solubilizer & | 5.00 | 0.01-30.00 |
| rolidone | Penetration | | |
| (Pharmasolve) | Enhancer | | |
| Propylene Glycol | Co-solvent | 20.00 | 1.00-80.00 |
| | for the | | |
| | solubilizer | | |
| Sorbitol (70% | Aqueous base | 3.00 | 1.00-50.00 |
| Non crystallizing) | | | |
| Polysorbate 60 | Non ionic | 1.50 | 0.01-10.00 |
| | Emulsifier | | |
| Purified Water | Aqueous Base | 52.25 | 1.00-80.00 |
| Total | | 100.00 | |

The composition of this example was prepared as follows:
1. Tacrolimus was dissolved in N-Methyl-Pyrrolidone and one by fourth quantity of Propylene Glycol was added to the solution and kept aside.
2. All of the ingredients in the oil phase were heated together to 70° C. to 75° C.
3. Polysorbate 60 was dispersed in Purified Water and heated to dissolve it.
4. The remaining quantities of Propylene Glycol and Sorbitol were added to the solution of step no. 3.
5. The solution of step no. 4 was heated to 70° C. to 75° C.
6. The hot solution from step no. 2 was added to the hot solution of step no. 5, slowly under continuous high speed stirring, to obtain a uniform cream base.
7. The cream base was allowed to cool to 40° C.
8. The tacrolimus solution of step no. 1 was added to the cream base of step no. 7 under gentle stirring.
9. After the addition was completed, the mixture was gently stirred for an additional 5 minutes to obtain a pharmaceutical cream composition having optimum and uniform consistency.

### EXAMPLE 3

### Cream—Formula Composition III

The ingredients used in this example are set forth below in Table 3.

TABLE 3

| | | | |
|---|---|---|---|
| | | | Range which |
| | | % w/w | can be used |
| Ingredients | Category | (in Example 3) | (% w/w) |

Oil Phase

| | | | |
|---|---|---|---|
| Stearic Acid | Oil Base | 13.00 | 5.00-40.00 |
| Stearyl Alcohol | | 1.00 | |
| Cetyl Alcohol | | 1.00 | |
| Methyl Paraben | Preservative | 0.10 | 0.01-2.00 |
| Propyl Paraben | Preservative | 0.05 | 0.01-2.00 |

Aqueous Phase

| | | | |
|---|---|---|---|
| Tacrolimus | API | 0.10 | 0.01-5.00 |
| (as Tacrolimus | | | |
| Monohydrate) | | | |
| N-Methyl Pyr- | Solubilizer & | 5.00 | 0.01-30.00 |
| rolidone | Penetration | | |
| (Pharmasolve) | Enhancer | | |
| Propylene Gly- | Co-solvent for | 20.00 | 1.00-80.00 |
| col | the solubilizer | | |
| Glycerin | Aqueous base | 10.00 | 1.00-50.00 |
| Potassium | Anionic Emul- | 0.90 | 0.01-10.00 |
| Hydroxide | sifier | | |
| Purified Water | Aqueous Base | 52.25 | 1.00-80.00 |
| Total | | 100.00 | |

The composition of this example was prepared as follows:
1. Tacrolimus was dissolved in N-Methyl-Pyrrolidone and one by fourth quantity of Propylene Glycol was added to the solution and kept aside.
2. All of the ingredients in the oil phase were heated together to 70° C. to 75° C.
3. Potassium Hydroxide was dissolved in Purified Water.
4. Remaining quantity of Propylene Glycol and Glycerin was added to the solution of step no. 3.
5. The solution of step no. 4 was heated to 70° C. to 75° C.
6. The hot solution from step no. 2 was added to the hot solution of step no. 5, slowly under continuous high speed stirring, to obtain a uniform cream base.
7. The cream base was allowed to cool to 40° C.
8. The Tacrolimus solution of step no. 1 was added to the cream base of step no. 7 under gentle stirring.
9. After the addition was completed, the mixture was gently stirred for an additional 5 minutes to obtain a pharmaceutical cream composition having optimum and uniform consistency.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

1. A pharmaceutical cream composition comprising (a) a therapeutically effective amount of one or more active pharmaceutical ingredients comprising one or more macrolide related immunosuppressants or pharmaceutically acceptable salts or esters thereof; (b) one or more cream forming agents; and (c) an effective amount of one or more skin penetration enhancers capable of percutaneous delivery of the macrolide related immunosuppressant through the skin.

2. The pharmaceutical cream composition of claim 1, wherein the macrolide related immunosuppressant is selected from the group consisting of tacrolimus, pimecrolimus, sirolimus, acsomycin, everolimus, pharmaceutically acceptable salts or esters thereof and mixtures thereof.

3. The pharmaceutical cream composition of claim 1, wherein the skin penetration enhancer is selected from the group consisting of one or more volatile organic solvents, one or more nonvolatile organic solvents and mixtures thereof.

4. The pharmaceutical cream composition of claim 3, wherein the volatile organic solvents are selected from the group consisting of a C₁-C₂₀ aliphatic, cycloaliphatic and/or aromatic-aliphatic alcohols, each of which is monohydric or polyhydric, alcohol/water mixtures, saturated or unsaturated fatty alcohols having about 8 to about 18 carbon atoms, saturated or unsaturated fatty acids or esters thereof having about 8 to about 18 carbon atoms and mixtures thereof.

5. The pharmaceutical cream composition of claim 3, wherein the volatile organic solvent is a C₁-C₂₀ alcohol.

6. The pharmaceutical cream composition of claim 3, wherein the non-volatile organic solvents are selected from the group consisting of pyrrolidones, polyol ethers, polyols and mixtures thereof.

7. The pharmaceutical cream composition of claim 6, wherein the pyrrolidones are selected from the group consisting of N-alkylpyrrolidones, vinyl pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones and mixtures thereof.

8. The pharmaceutical cream composition of claim 1, wherein the skin penetration enhancer is diethylene glycol monoethyl ether.

9. The pharmaceutical cream composition of claim 6, wherein the polyol ethers are a C₂-C₃₀ polyol ether containing from 2 to about 10 hydroxyl groups.

10. The pharmaceutical cream composition of claim 6, wherein the polyol ethers are selected from the group consisting of ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether and mixtures thereof.

11. The pharmaceutical cream composition of claim 6, wherein the polyols are selected from the group consisting of ethylene glycol, propylene glycol, butylene glycol, hexylene glycol, propylene glycol monocaprylate and mixtures thereof.

12. The pharmaceutical cream composition of claim 1, wherein the skin penetration enhancer comprises a pyrrolidone in at least one solvent selected from the group consisting of C₁-C₄ alcohols, C₁-C₄ alkylene glycols, C₁-C₄ polyalcohols, C₁-C₄ polyalkylene glycols and mixtures thereof.

13. The pharmaceutical cream composition of claim 1, comprising about 0.01% to about 5% w/w of the macrolide related immunosuppressant or pharmaceutically acceptable salt or ester thereof and about 0.01% to about 30% w/w of the skin penetration enhancer.

14. The pharmaceutical cream composition of claim 1, comprising about 0.02% to about 2% w/w of the macrolide related immunosuppressant or pharmaceutically acceptable salt or ester thereof and about I % to about 10% w/w of the skin penetration enhancer.

15. The pharmaceutical cream composition of claim 1, wherein the macrolide related immunosuppressant is tacrolimus or a pharmaceutically acceptable salt or ester thereof and the skin penetration enhancer is a polyol ether.

16. The pharmaceutical cream composition of claim 1, comprising about 0.01% to about 5% w/w of tacrolimus or a pharmaceutically acceptable salt or ester thereof and about 0.01% to about 30% w/w of a glycol ether.

17. The pharmaceutical cream composition of claim 1, comprising about 0.02% to about 2% w/w of tacrolimus or a pharmaceutically acceptable salt or ester thereof and about 1% to about 10% w/w of a glycol ether.

18. The pharmaceutical cream composition of claim 1, further comprising one or more pharmaceutically acceptable excipients.

19. The pharmaceutical cream composition of claim 1, further comprising one or more additional active pharmaceutical ingredients.

20. A process for preparing a pharmaceutical cream composition comprising:
(a) dissolving a therapeutically effective amount of one or more active pharmaceutical ingredients comprising one or more macrolide related immunosuppressants or pharmaceutically acceptable salts or esters thereof in one or more penetration enhancers to form a first solution;
(b) providing one or more cream forming phase comprising one or more cream forming agents; and
(c) mixing the product of step (a) with the product of step (b).

21. The process of claim 20, wherein the macrolide related immunosuppressant is selected from the group consisting of tacrolimus, pimecrolimus, sirolimus, acsomycin, everolimus, pharmaceutically acceptable salts or esters thereof and mixtures thereof.

22. The process of claim 21, wherein the skin penetration enhancer is selected from the group consisting of one or more volatile organic solvents, one or more nonvolatile organic solvents and mixtures thereof.

23. The process of claim 22, wherein the volatile organic solvents are selected from the group consisting of a C₁-C₂₀ aliphatic, cycloaliphatic and/or aromatic-aliphatic alcohols, each of which is monohydric or polyhydric, alcohol/water mixtures, saturated or unsaturated fatty alcohols having about 8 to about 18 carbon atoms, saturated or unsaturated fatty acids or esters thereof having about 8 to about 18 carbon atoms and mixtures thereof.

24. The process of claim 22, wherein the nonvolatile organic solvents are selected from the group consisting of pyrrolidones, polyol ethers, polyols and mixtures thereof.

25. The process of claim 24, wherein the pyrrolidones are selected from the group consisting of N-alkylpyrrolidones, vinyl pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones and mixtures thereof.

26. The process of claim 24, wherein the polyol ethers are selected from the group consisting of ethylene glycol monomethyl ether, ethylene glycol monoethyl ether, ethylene glycol monobutyl ether, ethylene glycol monopropyl ether, ethylene glycol monophenyl ether, ethylene glycol monohexyl ether, diethylene glycol monoethyl ether, diethylene glycol monomethyl ether, triethylene glycol monomethyl ether, triethylene glycol monoethyl ether, ethylene glycol monopropyl ether, ethylene glycol monobutyl ether, diethylene glycol monobutyl ether, triethylene glycol monobutyl ether, ethylene glycol monohexyl ether, diethyl glycol monohexyl ether, ethylene glycol phenyl ether.

27. The process of claim 24, wherein the polyols are selected from the group consisting of ethylene glycol, propylene glycol, butylene glycol, hexylene glycol, propylene glycol monocaprylate and mixtures thereof.

28. A method for delivering one or more macrolide related immunosuppressants or pharmaceutically acceptable salts or esters thereof through the skin in order to treat conditions situated on and beneath the skin comprising the step of topically administering an effective amount of a pharmaceutical cream composition comprising (a) a therapeutically effective amount of one or more active pharmaceutical ingredients comprising one or more macrolide related immunosuppressants or pharmaceutically acceptable salts or esters thereof; (b) one or more cream forming agents; and (c) an effective amount of one or more skin penetration enhancers capable of percutaneous delivery of the macrolide related immunosuppressant through the skin.

29. The method of claim 28, wherein the macrolide related immunosuppressant is selected from the group consisting of tacrolimus, pimecrolimus, sirolimus, acsomycin, everolimus, pharmaceutically acceptable salts or esters thereof and mixtures thereof.

30. The method of claim 28, wherein the pharmaceutical cream composition comprises about 0.01% to about 5% w/w of the macrolide related immunosuppressant or pharmaceutically acceptable salt or ester thereof and about 0.01% to about 30% w/w of the skin penetration enhancer.

31. The method of claim 28, wherein the pharmaceutical cream composition comprises about 0.02% to about 2% w/w of the macrolide related immunosuppressant or pharmaceutically acceptable salt or ester thereof and about 1% to about 10% w/w of the skin penetration enhancer.

32. The method of claim 28, wherein the macrolide related immunosuppressant is tacrolimus or a pharmaceutically acceptable salt or ester thereof and the skin penetration enhancer is a glycol ether.

33. The method of claim 28, wherein the pharmaceutical cream composition comprises about 0.01% to about 5% w/w of tacrolimus or a pharmaceutically acceptable salt or ester thereof and about 0.01% to about 30% w/w of a glycol ether.

34. The method of claim 31, wherein the pharmaceutical cream composition comprises about 0.02% to about 2% w/w of tacrolimus or a pharmaceutically acceptable salt or ester thereof and about 1% to about 10% w/w of a glycol ether.
